(19) United States
(12) Patent Application Publication
(10) Pub. No.: US 2017/0356867 A1
(43) Pub. Date: Dec. 14, 2017

(54) NANOSCALE SENSOR, SYSTEM TO MANUFACTURE THE SENSOR, AND METHOD TO MANUFACTURE THE SENSOR

(71) Applicant: Thomas Michael Daunais, Ann Arbor, MI (US)

(72) Inventor: Thomas Michael Daunais, Ann Arbor, MI (US)

(21) Appl. No.: 15/281,918

(22) Filed: Sep. 30, 2016

Related U.S. Application Data
(60) Provisional application No. 62/348,012, filed on Jun. 9, 2016.

(51) Int. Cl. G01N 27/04; H01L 21/3205; H01L 21/027; G01N 33/487; G03F 7/00; H01L 29/16; H01L 29/06; H01L 21/321

(52) U.S. Cl. CPC G01N 27/045; H01L 29/0669; H01L 21/32055; H01L 21/321; G01N 33/48707; G03F 7/0035; H01L 29/16; H01L 21/0273

(57) ABSTRACT

A nanoscale sensor, and method to manufacture the sensor. The sensor is designed to measure the change in free carriers from analyte detection by measuring current with an applied bias across the nano-wire(s) in a tested aqueous solution. The measured current is compared to known calibrated concentrations of the tested characteristic bacterium, virus, chemical, gas, or some combination thereof and a value for the tested aqueous solution. Temperature, pH and salinity measuring circuits are included to enable environmental correction.

### CROSS-REFERENCE TO RELATED APPLICATIONS

This application claims the priority benefit of U.S. Provisional Application 62/348,012 filed Jun. 9, 2016, which is herein incorporated by reference in its entirety.

### TECHNICAL FIELD

The present disclosure relates to nanoscale sensors. More particularly, nanoscale sensors designed for testing aqueous solutions.

### BACKGROUND

The statements in this section merely provide background information related to the present disclosure. Accordingly, such statements are not intended to constitute an admission of prior art.

To date, the diagnostics field stretching from food and water safety to medical diagnostics has limited ability to sense many analytes of interest. Of those that can be sensed currently, including but not limited to bacteria, viruses, and miRNA, many measurements require processing that may take multiple days and even up to one week to obtain conclusive results. Determining susceptibilities of several bacteria such as mycobacteria to antibiotics in many cases may require several days to ascertain. One of the most sensitive methods to identify analytes of interest currently available to the medical industry is a label detection method called fluorescent tagging. In this method, a fluorescent molecule is bound to a chemical that binds with the analyte of interest. After this bond is made, the sample is then put in an instrument such as a photomultiplier to detect the presence of the fluorescent tag. This procedure requires multiple processing steps to prepare the sample that is to be measured, allowing for the possibility of sample contamination. Even with this technique, it is not possible to detect many analytes of interest because the method lacks the ultimate resolution required for very low concentration detection. The key to the next generation of medical sensing technology is to increase the ultimate resolution of the testing method.

In 2012 in the United States, approximately 50 million people experienced a foodborne illness. A significant number of these illnesses were traced back to E. coli contamination of food. This resulted in an estimated 130,000 hospitalizations and 3,000 deaths. Organic farms, farmers markets, and food imports have added substantially to America's table to meet increased demand, but this expansion of food sources will require a matching ramp-up in inspections. In 2009, the FDA estimated 24 million shipments of FDA regulated goods passed through the nation's ports of entry, up from 6 million a decade earlier. During that time, the number of FDA investigators stayed constant at about 1,350. Antiquated food testing methods and overworked inspectors ensure that only a small percentage of the U.S. food supply is tested for pathogen contamination.

To help meet the regulation requirements for testing, many large food production houses perform in-house testing on their own products. Currently utilized testing methods require one week for conclusive results because they require slow laboratory based testing methods including culture growth prior to testing. Many of these tests occur at labs off-site from the 28,000 food processing facilities in the U.S., adding delay and cost. The industry would benefit from sensing devices that provide a shorter duration from test sample to result, and can be deployed on site.

### BRIEF SUMMARY OF THE INVENTION

A nano-device comprises: a sensor consisting of a semiconductor with features smaller than 1 micron; a contact junction, and/or contact region.

In one embodiment, the nano-device further comprises a pH sensor, a temperature sensor, and a salinity sensor. The pH and salinity sensors are combined using a shared counter-electrode.

In one embodiment, the salinity measurement is made by two parallel noble metal pads (e.g. gold, platinum) exposed to a test solution. A measurement is made by applying an AC or DC voltage bias between the two plates, measuring the current, and applying an algorithm developed for the specific size and spacing of the pads.

In one embodiment, the pH sensor is created by using a semi-conductor sensing region, coated in an ion-selective membrane such as polyaniline, and applying a voltage via counter electrode to push ions to the sensor. The counter electrode in this embodiment is one of the salinity sensor pads, located near the silicon minibar that is coated with polyaniline to create a sensor capable of detecting pH.

In one embodiment, the sensor can be an approximately 70 nm cross-section silicon nanowire manufactured using a method as described in international patent application PCT/US2014/065403. In one embodiment, the method cited in PCT/US2014/065403 comprises the steps of: depositing a silicon nitride layer on a silicon on insulator (SOI) starting wafer; patterning the silicon nitride to define at least one silicon microbar; etching the SOI starting wafer to expose the at least one silicon microbar, wherein the at least one microbar exists containing a raised perimeter; growing a silicon oxide layer on the raised perimeter of the at least one microbar; and etching a portion of the at least one silicon microbar to produce at least one silicon nanowire adjacent the silicon oxide layer.

The nanowire has a diamond cubic crystal structure that is the same or similar to a lattice structure in the contact junction. Hence, the contact region is a continuous and unbroken lattice structure going from the contact junction to the nanowire.

In one embodiment, the contact region that forms a continuous and unbroken lattice structure going from the contact region to the nano-wire is achieved using a material gradient from the bottom of a silicide to a lightly doped silicon. At the contact, the gradient starts with a metal (e.g. titanium), then transitions to a silicide, then transitions to gradient doped silicon, and then transitions to silicon.

In one embodiment, dopants are added to the silicon and a heat treatment drives the high concentration of dopants on the surface into the lower concentration region, resulting in a reduction of potential barriers set up between different materials. This results in an ohmic or ohmic-like junction, and a more repeatable device behavior.

In one embodiment, a first step in forming a continuous and unbroken lattice structure is to provide an area of gradient doping from the bulk device of 10 ohm/cm to degenerate doping at the silicided interface. This is done by an in-situ boron doped polysilicon deposition, and a subsequent short annealing process. Following this step, a small amount of metal is deposited, and rapidly heated to a temperature in which a eutectic material is formed (in our case, titanium to titanium silicide.) Following this step, another metal stack is deposited to form the adhesion and capping material used for the metal contact formation to the nano-scaled device.

In one embodiment, n-type dopants are added to the contact surface creating a junction which behaves as a p-n junction due to the differences in charge properties between the surface of the contact region and the body of the silicon sensor region. The p-n junction region results in highly repeatable electrical behavior from the resulting device.

In one embodiment, the contact region can be configured to maintain the lattice structure in a transition between a micro-scaled contact and the sensor.

In one embodiment, the junction comprises a metal-semiconductor blend wherein the metal concentration gradually decreases further from the surface. Materials of construction can be tantalum silicide or the like. The gradual decrease in concentration eliminates sharp junction properties (voltage potential). The device uses microscale contacts connected to a nanoscale sensor. This enables semi-classical bulk silicon electrical properties to predominate over the quantum mechanical properties caused by surface states and lattice strain in nanoscale semiconductor-metal junctions.

A method embodiment to manufacture the nano-device comprises: patterning a photoresist to cover only the contact regions of a nano-device; depositing a non-metal masking layer (such as silicon nitride) on the nano-device via a method such as sputter deposition (other methods possible); removing the photoresist via submersion in acetone; wherein the removal lifts off the unwanted masking material and leave a thin film that only exposes the contact areas; intrinsic poly-silicon deposition (to set up buffer dopant layer); highly doped poly-silicon deposition in the same tube as previous set to avoid oxygen exposure; removing the masking layer; patterning photoresist to expose regions and pad areas only; depositing a metal layer on the nano-device; removing the photoresist and metal from all regions of the nano-device except the contact areas and pads; and sintering the metal to the highly doped silicon with a rapid thermal process.

A method embodiment to manufacture the nano-device comprises: patterning a photoresist to cover only contact regions and pad areas of a nano-device; depositing a non-metal masking layer on the nano-device; removing the photoresist via a submersion in acetone, wherein the removal lifts off any unwanted masking material not required by the desired pattern and leaves a thin film that only exposes the contact regions and pad areas; depositing intrinsic poly-silicon in a tool that pulls a vacuum to remove oxygen to set-up a buffer dopant layer; depositing highly doped poly-silicon in the same tube as previous set utilizing the same tool used to deposit the intrinsic polysilicon without removing the nano-devices sensor from the vacuum state established in the previous deposition to avoid oxygen exposure; removing the masking layer; patterning a photoresist to expose the contact regions and pad areas only; depositing a metal layer on the nanoscale sensor; removing the photoresist and any unwanted metal from all regions of the nano-device except the contact regions and pad areas; and sintering the metal layer to the highly doped silicon with a rapid thermal process.

The scope of the invention is defined by the claims, which are incorporated into this section by reference. A more complete understanding of embodiments on the present disclosure will be afforded to those skilled in the art, as well as the realization of additional advantages thereof, by consideration of the following detailed description of one or more embodiments. Reference will be made to the appended sheets of drawings that will first be described briefly.

### BRIEF DESCRIPTION OF THE DRAWINGS

A clear understanding of the key features of the invention summarized above may be had by reference to the appended drawings, which illustrate the method and system of the invention, although it will be understood that such drawings depict preferred embodiments of the invention and, therefore, are not to be considered as limiting its scope with regard to other embodiments which the invention suggests. Accordingly:

FIG. 1A shows a cross-section of a nano-device.

FIG. 1B shows a more detailed cross-section of a nano-device.

FIG. 2 shows the crystalline lattice structure of the entire device.

FIG. 3 shows the crystalline structure of the transition region.

FIG. 4 shows a circuit diagram with an embedded temperature sensor, pH sensor, and salinity sensor.

FIG. 5 is a chart that shows nanowire repeatability of output current for nanowires held at a 5 volt bias.

FIG. 6 is a flowchart showing the steps required to make a nanowire embodiment.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present disclosure discusses a nanotechnology configured to solve the needs discussed in the background. The technology consists of a nano-device, system to manufacture the nano-device, and method to manufacture the nano-device.

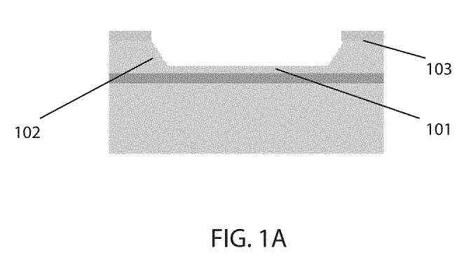

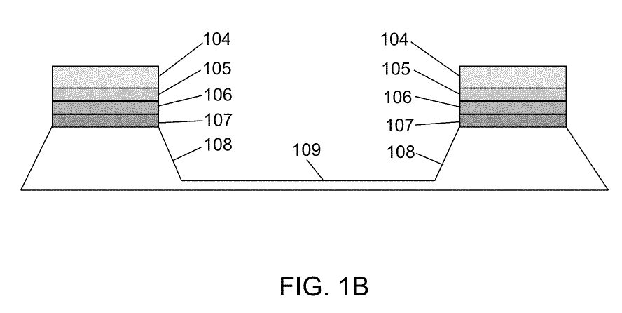

FIG. 1A shows a cross-section of a nano-device. Shown are a sensor 101, contact region 102, and junction 103.

FIG. 1B shows a more detailed cross-section of a nano-device. Shown are capping metal 104, titanium 105, titanium silicide 106, gradient doped silicon 107, original crystalline silicon 108, and silicon nanowire 109.

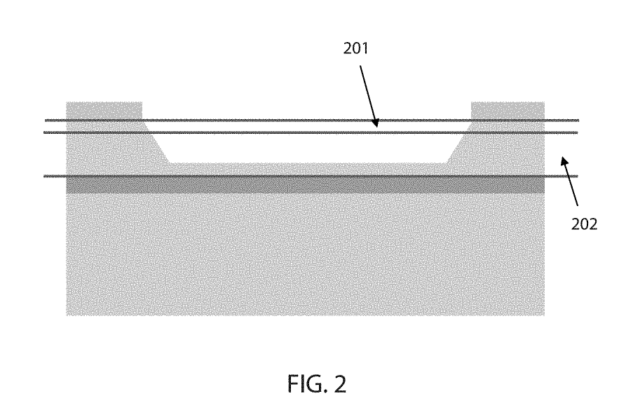

FIG. 2 shows the areas of constant crystalline lattice structure of the entire device. Shown are a first lattice structure 201 and a second lattice structure 202.

For the first lattice structure 201: one of the key pieces of the invention is the consistency of the lattice structure that is maintained between the nano-scaled dimensions of the physical features of the device (in this case a nanowire sensor) and the micro-scaled contact region. By keeping the lattice structure a constant between the nano and micro-scaled regions of the device, electronic states that would create trap sites near the sensor region are kept to a minimum; this in turn means more predictable electronic behavior and better uniformity between devices in manufacturing.

For the second lattice structure 202: the diamond cubic lattice structure of silicon is not consistent due the sintering process in which the deposited metal layer is infused into the silicon. This creates a gradient of the metal material in the silicon, which helps to eliminate sharp junctions and surface states between the two materials.

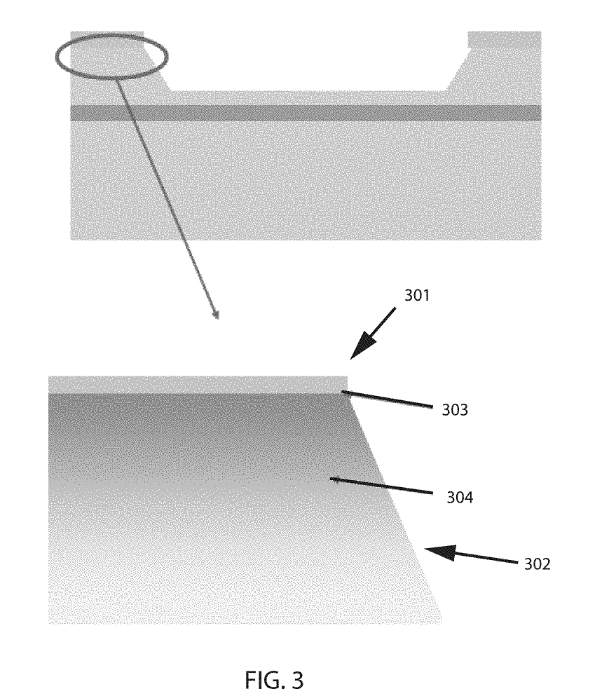

FIG. 3 shows the material make up of the contact region. Shown are a metal layer 301 and a silicided region 302 on top of single crystalline silicon sensor 303. The metal layer 301 is deposited on top of the contact metal 302 after the heat treatment process is completed which drives the first metal into the semiconductor device below forming a silicide material. The contact silicide 302 has a gradient concentration of metal, highest at the interface 303 with the metal 301 and lowest at the sensors crystal silicon 304. This contact method removes the overwhelming control of surface states on the junction.

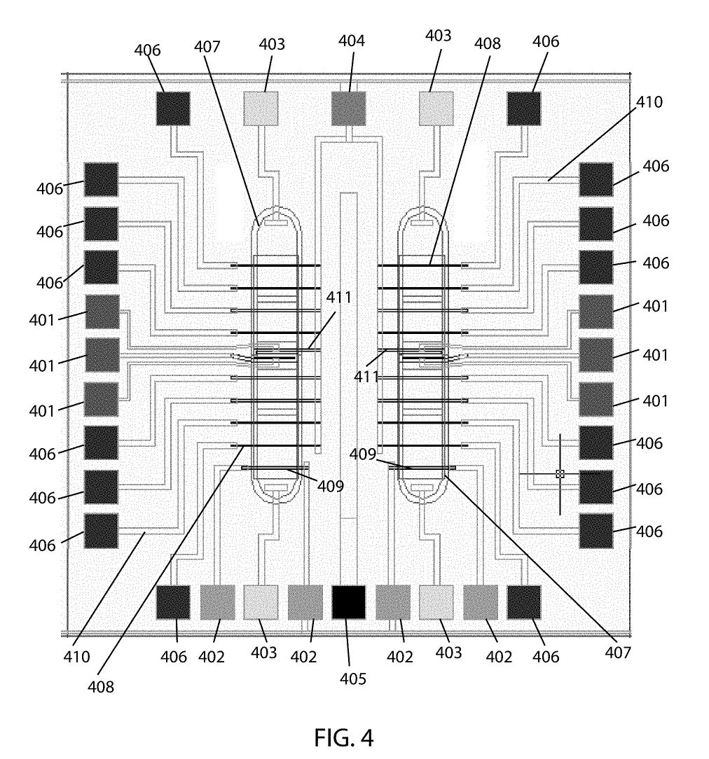

FIG. 4 shows a circuit diagram, giving a top-down view of a nano-device embodiment, with an embedded temperature sensor, pH sensor, and salinity sensor. Contact pads for the various sensors are shown as squares. Shown are contacts for temperature sensor 401, pH sensor 402, salinity sensor 403, common ground 404, backgate 405 which provides a contact to the silicon underneath, nanowire sensors 406, a hockey-rink shaped area 407 in which a measured fluid is placed, silicon nanowires 408, silicon microbars 409 for pH sensing, individual wires 410 connecting contact pads to nanowires 408, microbars 409, or directly to the hockey-rink shaped area 407, and serpentine silicon microbars 411 for temperature sensing. Each set of three temperature sensor 401 pads is combined with a resistance measurement to create a 4 point single temperature measurement. For the salinity sensor 403, current travels through the measured fluid to acquire a measurement.

Each nanowire sensor 406 corresponds to a measured characteristic such as a bacterium, virus, chemical, or gas (or some combination thereof). An algorithm for the measured characteristic(s) is/are determined beforehand by testing a known controlled amount and establishing corresponding sensor values. The sensors measure the change in free carriers by measuring current with an applied bias across the nano-wire. That is, apply two voltages and look at current compared to the algorithm (calibration curve).

An encapsulant such as epoxy, silicone, or some other non-conductive material is used to prevent short-circuiting the sensor. The application is controlled by the hockey-rink shaped structures 407 to set up testing channels.

The pH sensor is electrically independent of the ground. A novel feature of the circuit shown in FIG. 4 is the combined functionality of pH and salinity, utilizing the same sample on the same circuit. The salinity sensor creates a bias for the pH sensor.

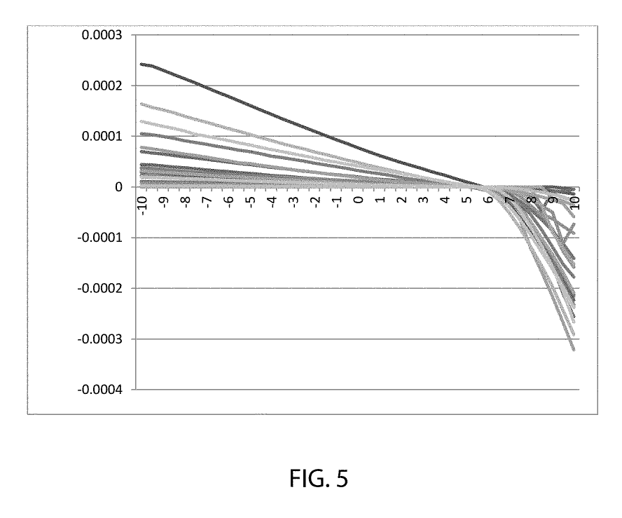

FIG. 5 is a chart that shows nanowire repeatability of output current for nanowires held at a 5 volt bias. Silicon nanowire sensing technology has been around for over a decade, and provides unparalleled device performance in both resolution and multiplexing technologies. These two unique properties have not been commercially utilized however due to issues with device repeatability. The below graph shows the electrical performance of 25 nanowires scanned across wafer lots with the vertical axis labeling output current across a nanowire held at 5 volt bias, and the horizontal axis shows the applied gate bias in respect to ground on the nanowire.

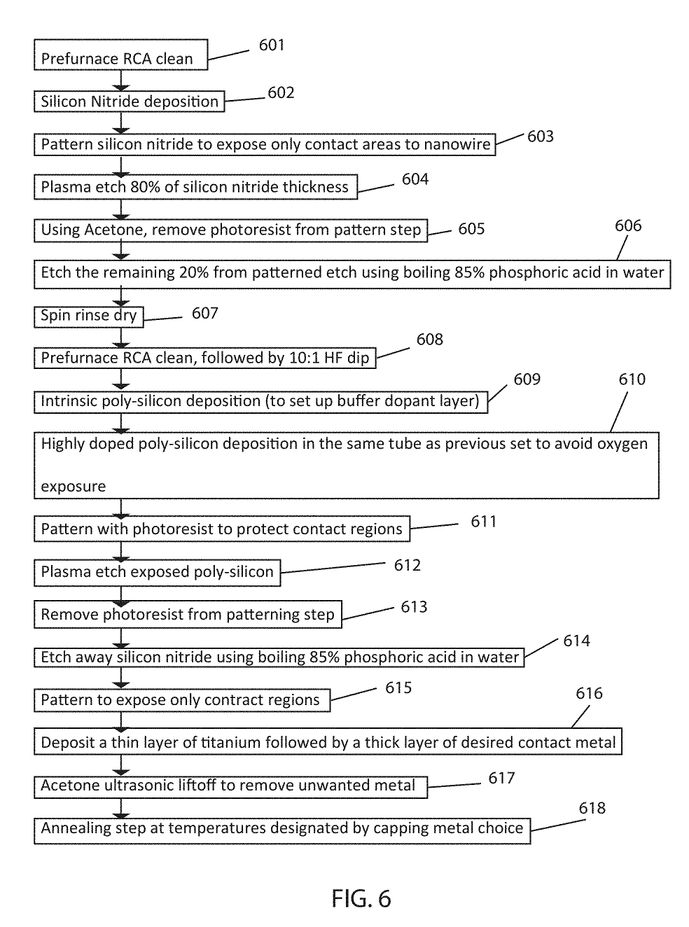

The repeatable performance shown in FIG. 5 is caused by a fundamental change in the way to contact the silicon nanowire. Instead of creating ohmic contacts, a junction region is purposefully set up using the attached process flow described in FIG. 6. The result is a transistor like device, with the clear junction characteristics shown in FIG. 5.

FIG. 6 is a flowchart showing the steps required to make a nanowire contact embodiment. Shown are step 601 Prefurnace RCA clean; step 602 Silicon Nitride deposition; step 603 Pattern silicon nitride to expose only contact areas to nanowire; step 604 Plasma etch 80% of silicon nitride thickness; step 605 Using Acetone, remove photoresist from pattern step; step 606 Etch the remaining 20% from patterned etch using boiling 85% phosphoric acid in water; step 607 Spin rinse dry; step 608 Prefurnace RCA clean, followed by 10:1 HF dip; step 609 Intrinsic poly-silicon deposition (to set up buffer dopant layer); step 610 Highly doped poly-silicon deposition in the same tube as previous set to avoid oxygen exposure; step 611 Pattern with photoresist to protect contact regions; step 612 Plasma etch exposed poly-silicon; step 613 Remove photoresist from patterning step; step 614 Etch away silicon nitride using boiling 85% phosphoric acid in water; step 615 Pattern to expose only contract regions; step 616 Deposit a thin layer of titanium followed by a thick layer of desired contact metal; step 617 Acetone ultrasonic liftoff to remove unwanted metal; and step 618 Annealing step at temperatures designated by capping metal choice.

All patents and publications mentioned in the prior art are indicative of the levels of those skilled in the art to which the invention pertains. All patents and publications are herein incorporated by reference to the same extent as if each individual publication was specifically and individually indicated to be incorporated by reference, to the extent that they do not conflict with this disclosure.

While the present invention has been described with reference to exemplary embodiments, it will be readily apparent to those skilled in the art that the invention is not limited to the disclosed or illustrated embodiments but, on the contrary, is intended to cover numerous other modifications, substitutions, variations, and broad equivalent arrangements.

I claim:

1. A method of manufacturing a nanoscale sensor, the method comprising:
patterning a photoresist to cover only contact regions and pad areas of a nano-device;
depositing a non-metal masking layer on the nano-device;
removing the photoresist via a submersion in acetone, wherein the removal lifts off any masking material not required by a desired pattern and leaves a thin film that only exposes the contact regions and pad areas;
depositing intrinsic poly-silicon in a tool that pulls a vacuum to remove oxygen to set-up a buffer dopant layer;
depositing highly doped poly-silicon utilizing the tool used to deposit the intrinsic polysilicon without removing the nano-device from the vacuum;
removing the masking layer;
patterning a photoresist to expose the contact regions and pad areas only;
depositing a metal layer on the nanoscale sensor;
removing the photoresist and any unwanted metal from all regions of the nano-device except the contact regions and pad areas; and
sintering the metal layer to the highly doped silicon with a rapid thermal process.

2. A nanoscale device, the device comprising:
a pH sensor;
a temperature sensor;
a salinity sensor, wherein the pH sensor and salinity sensor are combined by using a shared counter-electrode;
one or more silicon nanowire sensor(s);
a contact junction; and
one or more contact region(s).

3. The nanoscale device of claim 2, wherein the salinity sensor further comprises two parallel noble metal pads.

4. The nanoscale device of claim 3, wherein the salinity sensor is calibrated by exposing the parallel noble metal pads to a test solution, applying an AC or DC voltage between the pads, measuring the current, and applying an algorithm developed for the a specific size and a specific spacing of the pads.

5. The nanoscale device of claim 2, wherein the pH sensor has a sensing region comprising a semi-conductor and is coated and is coated in an ion-selective membrane.

6. The nanoscale device of claim 5, wherein the counter-electrode is configured to accept a voltage and force ions to the pH sensor.

7. The nanoscale device of claim 2, wherein the silicon nanowire sensor(s) are approximately 70 nm in diameter.

8. The nanoscale device of claim 2, wherein the silicon nanowire sensor(s) have a lattice structure that is similar to a lattice structure of the contact region(s).

9. The nanoscale device of claim 2, wherein there is a continuous and unbroken lattice structure between the silicon nanowire sensor(s) and a respective contact region(s), further wherein the unbroken lattice structure is achieved by using a gradient from titanium to silicon.

10. The nanoscale device of claim 2, wherein the silicon nanowire sensor(s) further comprise heat-treated dopants.

11. The nanoscale device of claim 2, wherein the silicon nanowire sensor(s) further comprise n-type dopants.

12. The nanoscale device of claim 2, wherein the nanoscale device is manufactured by utilizing the method of claim 1.

13. The nanoscale device of claim 12, wherein the salinity sensor further comprises two parallel noble metal pads.

14. The nanoscale device of claim 13, wherein the salinity sensor is calibrated by exposing the parallel noble metal pads to a test solution, applying an AC or DC voltage between the pads, measuring the current, and applying an algorithm developed for the a specific size and a specific spacing of the pads.

15. The nanoscale device of claim 12, wherein the pH sensor has a sensing region comprising a semi-conductor and is coated and is coated in an ion-selective membrane.

16. The nanoscale device of claim 15, wherein the counter-electrode is configured to accept a voltage and force ions to the pH sensor.

17. The nanoscale device of claim 12, wherein the silicon nanowire sensor(s) are approximately 70 nm in diameter.

18. The nanoscale device of claim 12, wherein the silicon nanowire sensor(s) have a lattice structure that is similar to a lattice structure of the contact region(s).

19. The nanoscale device of claim 12, wherein there is a continuous and unbroken lattice structure between the silicon nanowire sensor(s) and a respective contact region(s), further wherein the unbroken lattice structure is achieved by using a gradient from titanium to silicon.
